(19) United States
(12) Patent Application Publication
(10) Pub. No.: US 2009/0088733 A1
(43) Pub. Date: Apr. 2, 2009

(54) Methods and apparatus for treatment of ocular melanosis

(76) Inventors: Richard Rox Anderson, Boston, MA (US); Hirotaka Akita, Aichi (JP)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 11/547,241

(22) PCT Filed: Apr. 4, 2005
(86) PCT No.: PCT/US05/11438

§ 371 Date: Nov. 17, 2008

Related U.S. Application Data
(60) Provisional application No. 60/559,102, filed on Apr. 3, 2004.

(51) Int. Cl. A61F 9/008; A61B 18/20

(52) U.S. Cl. 606/4

(57) ABSTRACT

The invention entails a novel system and method for modifying scleral pigmentation. More particularly, the invention entails a novel system and method for treating Ocular Melanosis (OM). The invention contemplates a system and method for treating OM that uses lasers with modified pulse widths and low power to whiten the sclera without causing damage to the retina. Ultimately, a system has been developed that uses of low laser pulse fluences considerably less than those used for treating nevus of Ota on the skin. The system limits the risk of retinal injury by use of (a) a small exposure spot size and (b) a high numerical aperture (NA) optical beam with fluences of between about 0.5 J/cm² and 5.0 J/cm², significantly lower than many currently available laser treatments. In a preferred embodiment, the system of the invention uses a laser with a wavelength in the visible and near-infrared spectrum and a pulse width from about 1 ns-100 microseconds.

### FIELD OF THE INVENTION

This invention relates in general to systems for and methods of treating ocular hyperpigmentation. More particularly this invention relates to hyperpigmentation of the sclera. Most particularly this invention describes systems and methods for the treatment of ocular melanosis.

### BACKGROUND OF THE INVENTION

Ocular melanosis (OM) is a disease that manifests as variable hyperpigmentation of the sclera of the eye and is often related to more common disorder—nevus of Ota. OM was first described in 1917 as a variable hyperpigmentation of the sclera, uvea and optic nerve (Bourquin J. Die angeboren Melanose des Auges. Ztschr. F. Augenh. 37 (supple) 294-311 (1917)).

A related condition, nevus of Ota is a congenital pigment disorder usually involving the skin of the face in areas supplied by the first and second branches of the trigeminal nerve. Patients with nevus of Ota have increased amounts of melanin (pigment) and melanin producing cells (melanocytes) in and around their eyes. Regions of hyperpigmentation include the intraocular blood vessel layer called the uvea (choroid, ciliary body and iris), on top of the white part of the eye ball (the episclera), and the eye lids. (Fitzpatrick T B, Zeller R Z, Kukita A, et al. Ocular and dermal melanocytosis. Arch Opthalmol, 52: 922-924 (1968)). Nevus of Ota, as a disorder, is relatively more common among Asians, and in particular, Asian females although it does present in Caucasians and Negroes.

The incidence of OM in patients with nevus of Ota is between 37% and 70% and comprises about 1% of all dermatological cases in Japan. All cases of OM are considered to be congenital (Kawamoto K. Clinical and histopathological studies of ocular melanosis. J. Jpn PRS., 13: 198-214 (1993)).

In addition to the social stigma many OM patients suffer because of the cosmetic aspects of the condition, they are at risk for a number of other conditions including malignant melanoma and glaucoma. In particular, patients with nevus of Ota are at greater risk for the development of intraocular and central nervous system malignant melanomas (e.g. choroidal melanoma). Intraocular melanomas although in these patients than the general population, still only occur in less than 4% of cases. About 10% of OM patients develop increased intraocular pressure, and some develop glaucoma. In Caucasians, ocular melanomas have been known to develop in patients with nevus of Ota and OM.

Until now, treatment options for OM have been extremely limited. Only one type of treatment has been currently proposed for OM. It involves a microsurgical technique, requiring tissue removal of the sclera. After this treatment, some of the patients developed ugly looking results and scars (Kawamoto K., Miyanaga Y., Suzuki T. et al., The microsurgical treatment of ocular melanosis, JPN. J. PR. S, 34: 813-818 (1991)).

Furthermore, currently accepted laser treatments, such as those used to treat nevus of Ota, cannot be used to treat OM. Dermal melanocytosis associated with nevus of Ota can and has been treated with Q-switched lasers using selective photothermolysis. (Ueda S, Isoda M, Imayama S. Response of nevus of Ota to Q-switched ruby laser treatment according to lesion colour, Br J Dermatol, 142: 77-83 (2000); Kono T, Nozaki M, Chan H H, wt al., A retrospective study looking at the long-term complications of Q-switched ruby laser in the treatment of Nevus of Ota, Lasers Surg. Med., 29:156-159 (2001); Taylor C R, Flotte T J, Gange R W, Anderson, R R., Treatment of Nevus of Ota by Q-switched ruby laser, J. Am. Acad Dermatol., 30: 743-751 (1994)).

Specifically, nevus of Ota is generally treated with red or near-infrared Q-switched lasers, which selectively kill pigmented cells distributed throughout the dermis. Although highly selective, the mechanism of pigmented cell injury during laser treatment for nevus of Ota is violent. These lasers are high powered and use short nanosecond pulses with fluences between 5-10 J/cm². Sudden heating of melanosomes at rates of about 10¹⁰° C./second is produced, with associated fracture, cavitation, and shock wave production. While highly effective, safe, and well tolerated in skin, such a procedure if used to treat a scleral hyperpigmentation or OM could potentially cause damage to the underlying retina and choroids layers of the eye; ultimately resulting in damage to the eye and even blindness.

Ocular melanosis is usually a benign condition that causes disfigurement but does not threaten visual function. Since currently available laser treatments may potentially injure the retina and choroidal layers of the eye underlying the sclera, such treatment has been deemed inappropriate for use in OM.

What is needed, therefore, is a novel, effective, safe and relatively scar free system and method of treatment for OM that does not have the same risks as currently available therapies.

### SUMMARY OF THE INVENTION

The invention entails a novel system and method for modifying scleral pigmentation. More particularly, the invention entails a novel system and method for treating Ocular Melanosis (OM). Most particularly, the invention is a laser-based system and method for treating OM.

Even more specifically, the invention contemplates a system and method for treating OM that uses lasers with modified pulse widths and low power to treat the eye. The invention describes a range of pulse widths and power that provide for whitening of the sclera without causing damage to the retina.

Ultimately, a system has been developed that uses low laser pulse fluences considerably less than those used for treating nevus of Ota on the skin. In addition the system limits the risk of retinal injury by use of (a) a small exposure spot size and (b) a high numerical aperture (NA) optical beam with fluences of between about 0.5 J/cm² and 5.0 j/cm², significantly lower than many currently available laser treatments. In a preferred embodiment, the system of the invention uses a laser with a wavelength in the visible and near-infrared spectrum and a pulse width from about 1 nanosecond-100 microseconds. In a most preferred embodiment of the invention the system contemplates a laser with a pulse width of about 1 microsecond and wavelength in the blue visible spectrum.

### BRIEF DESCRIPTION OF THE DRAWINGS

FIG. 1A: Type I pigmentation, seen grossly as an isolated pigmented macula on the pig sclera.

FIG. 1B: Scleral melanocytes of type I pig eyes are located in a superficial layer of the scleral stroma (Fontana-Masson stain).

FIG. 2A: Type II pigmentation, seen grossly as nearly confluent small dark brown pigment all over the sclera.

FIG. 2B: Scleral melanocytes of type II pig eyes are located throughout the sclera (hematoxylin and eosin stain).

FIG. 3: Porcine sclera melanocytes are round or elliptical cells which contain many 0.3-1.5 μm melanosomes, most of which are mature (stage IV).

### DETAILED DESCRIPTION OF THE INVENTION

In order to develop and test a system for addressing OM, an animal model with ocular characteristics similar in thickness and pigmentation to humans with nevus of Ota was needed. Although there are many articles on retinal pigment epithelium (RPE) cell disorders amongst different animals that serve as models for studies of human retinal diseases and treatments, models of scleral pigmentation similar to human ocular melanosis have not been reported. Scleral pigmentation in pig eyes is similar to that described for the superficial and deep types of human ocular melanosis. As such, pigs appear to be an appropriate animal model for developing systems and methods for the treatment of human ocular melanosis. Ultimately, a scleral pigmentation model in an outbred, farm pig was used.

The distribution of scleral melanin in outbred farm pig eyes by light microscopy, and the distribution and characteristics of scleral melanosomes by electron microscopy was determined. Two types of scleral pigmentation were found, corresponding to superficial and full-thickness distribution of scleral melanocytes. Both types provide a useful animal model for human ocular melanosis, which also occurs in superficial and deep variants.

Medical lasers having a range of pulse widths, wavelengths and power (fluences) were tested on the outbred farm pig model. As more fully described below, a relatively low power laser pulse in the visible spectrum was found to be effective while limiting the potential for retinal damage.

### EXAMPLE 1

### Animal Model for Ocular Melanosis

Ten freshly enucleated outbred farm pig eyes were obtained from a local abattoir. The eyes were examined, photographed, dissected and samples of sclera were immediately immersed in 10% buffered formalin, which were routinely processed and embedded in paraffin. Four to six micrometer thick sections were cut and stained with hematoxylin and eosin (H&E) as well as Fontana-Masson (FM) stains. Tissue samples were also obtained for electron microscopy (EM). These were immersed in Karnovsky's solution, and minced into approximately 1 mm cubes. After 5 h of fixation, these were rinsed with 0.1 M sodium cacodylate buffer, pH 7.4 for 2 h, then postfixed with 2% osmium tetroxide for 2 h. After two 15 minute rinses in 0.1 M sodium cacodylate buffer, the tissue was dehydrated in graded ethanol solutions and embedded in Epon. Ultra thin 1 μm sections, were cut on a Porter-Blum MT2-B ultramicrotome (Dupont Inst., Newton, Conn.), and examined with a Zeiss EM109 transmission electron microscope (Carl Zeiss, Inc., Oberkochen, West Germany).

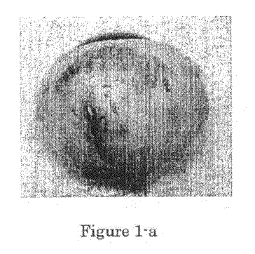

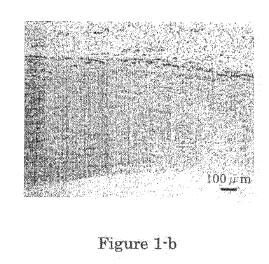

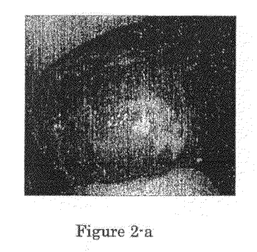

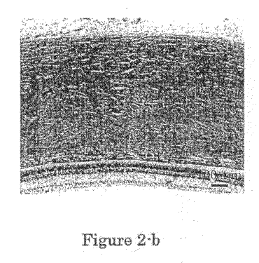

Two patterns of scleral pigmentation were found, and the eyes were divided into two groups according to the type of pigmentation. In type I, scleral pigmentation appears as an isolated macula (FIG. 1-a), and in type II as small confluent flecks of dark brown pigment all over the sclera (FIG. 2-a). Each group consisted of five eyes.

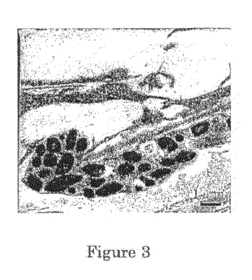

By light microscopy, eyes with an isolated pigmented macula (type I) had melanocytes confined to a superficial layer of the scleral stroma (FIG. 1-b), extending typically 100 μm deep. In the speckled type (type II), melanocytes were located in both the superficial and deep layers of the sclera (FIG. 2-b). Scleral melanocytes were not seen in non-pigmented areas of the sclera. By transmission electron microscopy, scleral melanocytes of both eye types were round or elliptical cells, containing cytoplasmic membrane-bound, melanosome organelles. The diameter of melanosomes ranged from 0.3-1.5 μm. Most of the melanosomes were mature (stage IV) and heavily pigmented (FIG. 3).

In a report of surgical approach for treatment, human ocular melanosis was studied by light and electron microscopy. Distribution of scleral melanocytes in human ocular melanosis associated with nevus of Ota, was classified as 1) superficial type, 2) deep type, and 3) diffuse type (superficial and deep). All cases were either superficial or diffuse type. This distribution corresponds well to the pig eye types I and II in our study. By electron microscopy, human melanosomes of ocular melanosis were 0.5 to 2.5 μm in diameter, and mostly in stage IV. This is similar to the ultrastructural size range (0.3-1.5 μm) and stage that was found for pig sclera melanosomes.

### EXAMPLE 2

### Laser Treatment of Melanosis

A. Q-Switch Lasers

Developing a system for treating aberrant scleral pigmentation generally, and OM in particular, requires that one create a system that provides for whitening of the sclera without causing damage to the retina. In particular, one needs to develop a laser-based system with modified pulse widths and low power.

Generally, lasers may be continuous wave (CW) or pulsed. A CW laser emits a continuous stream of light as long as the medium is excited. A pulsed laser will emit light only in pulses, which may vary from femtoseconds (quadrillionths of a second) to seconds. The simplest way to pulse a laser is to use a mechanical shutter, similar to that in a camera, which works down to the millisecond range. Flashlamps (similar to those used in photographic strobe lights) can also be used to produce low millisecond range pulses. Pulses in the micro- to nanosecond range are produced using Q-switching.

In Q- (or Quality) switching, a crystal which rotates the polarization of light with very short pulses of applied high-voltage, called a Pockels Cell, is placed in the laser cavity in front of the reflective mirror, with a suitable polarizing filter to block excited photons. The laser medium can be maximally excited, and when voltage is applied to the Pockels Cell, its polarization rotates to match that of the polarizing filter, and photons pass through to the mirror to stimulate a very short, very high energy laser pulse

Q-switching a pulsed laser increases its peak power (P=E/t), shortens the output pulse width, and improves the consistency of the output power from pulse to pulse. However, there is almost always a net reduction in the average output energy compared to free-running pulsed mode. In free-running pulsed laser operation, lasing starts as soon as there is enough of a population inversion to trigger stimulated emission and resonance within the laser cavity (resonator). Each pulse will be somewhat different in total power and shape than every other (which is why pulsed laser power output is expressed in joules rather than watts). In effect, a Q-switch disables the laser resonator until the population inversion is complete.

The easiest way to disable a laser resonator is to block the path to one of the mirrors. In this instance, the medium is pumped, but there can be no stimulated emission until the Q of the resonant cavity is restored, resulting in a brief but intense pulse of laser energy. Q-switching can be accomplished simply by taking a mirror (typically the HR) out of alignment, usually by mechanically rotating the mirror. Most medical lasers use an electro-optical Q-switch (a Kerr or Pockel Cell) placed in the path of the beam within the resonator. In this instance a delay circuit opens the switch a preprogrammed time after the rod is pumped. A third method is to use a saturable absorber as a “passive” Q-switch. Below a certain threshold, these materials block light below a certain threshold, preventing the cavity from resonating. Above that threshold, the material becomes optically transparent to the particular laser wavelength, allowing lasing to occur. This process can repeat itself producing a series of ultrashort, high power laser pulses.

High energy, ultrashort pulses of laser light cause extremely rapid heating of the target, with formation of a rapidly expanding thermal plasma. As the plasma collapses, the shock wave causes mechanical disruption of the target. This photomechanical disruption is utilized by Q-switched lasers to treat tattoos and certain pigmented skin lesions such as nevus of Ota.

Q-switched lasers have never been used to address ocular melanosis, however. Q-switched laser selective photothermolysis has, however, been recently employed as a treatment for open-angle glaucoma, in a procedure called selective laser trabeculoplasty (SLT). (Latina M A, Sibayan S A, Shin D H, Noecker R J, Marcellino G. Q-switched 532-mm Nd:YAG laser trabeculoplasty (selective laser trabeculoplasty): a multicenter, pilot, clinical study, 105: 2082-2088 (1998); Park C H, Latina M A, Schuman J S. Developments in laser trabeculoplasty. Ophthalmic Surg Lasers, 31: 315-22 (2000); Latina M A, Tumbocon J A. Selective laser trabeculoplasty: a new treatment option for open angle glaucoma, Curr Opin Opthalmol, 13: 94-96 (2002)).

In SLT, Q-switched green laser pulses are used to target pigmented phagocytic cells in the trabecular meshwork, which participate in governing the outflow of aqueous humor. These cells are related to macrophages, not melanocytes, and are pigmented due to phagocytosis of melanin-containing debris. The primary advantage of SLT over conventional argon-ion laser trabeculoplasty, is that SLT does not destroy the supporting connective tissue of the trabecular meshwork. SLT is presently the only example of a Q-switched laser treatment used to target pigmented cells in the eye. As with Q-switched laser treatment of nevus of Ota, localized cavitation, fracture of pigment granules, and disruption of the target cells occurs. The safety of SLT suggests that it may be possible to treat ocular melanosis due to nevus of Ota safely, as well. The pig eye model may be useful for study of this and other approaches for treatment of ocular melanosis.

B. Scleral Pigmentation Removal System

Table I sets forth the details of the experiment. Four different wavelength Q-switched lasers were used to treat samples of six (6) pig eyes of the model developed in Example 1. Pig eyes with both type I and type II scleral pigmentation were used. Spot sizes and pulse durations varied for each laser. The eyes were treated with powers that ranged from 0.5 J/cm² to 5 J/cm². The eyes were examined for immediate whitening and retinal damage.

Immediate whitening is an immediate change in the blue-gray color of the sclera in a subject with aberrant scleral pigmentation or ocular melanosis, to white. An identical response is seen with Q-switched laser treatment of pigmented skin lesions including nevus of Ota in skin. Immediate whitening is due to the formation of small gas bubbles. The whitening fades away slowly over time as the gas bubbles dissolve into the tissue.

Retinal damage was assessed by light microscopic examination of stained sections from animal eyes after exposure of the sclera to laser pulses. Hematoxylin and eosin staining, and nitroblue tetrazolium chloride staining, were used to assess structural damage and thermal necrosis of retina, respectively.

The following currently commercially available lasers were used: (a) Q-Alex: ALEX LAZR™ (Candela Corporation); (b) Q-ruby: Spectrum™ RD-1200 Q-switched ruby laser (Palomar Medical Technologies); (c) Q-YAG: Palomar Q-YAG 5™ (Palomar Medical Technologies; (d) A 488 nm Dye laser (Palomar Medical Technologies). In order to achieve the appropriate power, the light from these lasers was attenuated by a variable number of thin glass plates, which partially transmit the laser beam. The pulse energy striking the tissue was adjusted by this attenuation, and by adjusting the electrical pump energy of the laser.

TABLE 1
Effectiveness of Varied Wavelength at Various Powers for Scleral Whitening

| | | | | | Immediately | Histological | |
|---|---|---|---|---|---|---|---|
| | | Pulse | | Energy | Whitening | damage in | |
| | Spot size | duration | Power | (mJ) | Phenomenon (IWP) | retina (H-E) | others |
| Dye | 1 mm | 1 μsec | 0.5 J/cm² | 3.925 | − | Not Done | |
| (488 nm) | | | | | | | |
| | | | 1 | 7.85 | +/− | − | |
| | | | 2 | 15.7 | + | − | minimal IWP: 9.5 mJ |
| | | | 3 | 23.55 | + | − | |
| | | | 4 | 31.4 | + | − | |
| | | | 5 | 39.25 | + | +/− | retina whitening |
| Q-GRN | 2 mm | 10 nsec | 0.5 J/cm² | 15.7 | − | ND | |
| (532 nm) | | | | | | | |
| | | | 1 | 31.4 | − | Not Done | |
| | | | 2 | 62.8 | − | ++ | minimal IWP: 117 mJ |
| | | | 3 | 94.2 | +/− | ++ | |
| | | | 4 | 125.6 | + | Not Done | |
| | | | 5 | 157 | + | Not Done | |
| Q-Ruby | 2 mm | 20 nsec | 0.5 J/cm² | 15.7 | +/− | +/− | |
| (694 nm) | | | | | | | |
| | | | 1 | 31.4 | + | +/− | |
| | | | 2 | 62.8 | + | ++ | |
| | | | 3 | 94.2 | + | ++ | |
| | | | 4 | 125.6 | + | Not Done | |
| | | | 5 | 157 | + | Not Done | |
| Q-Alex | 2 mm | 50 nsec | 0.5 J/cm² | 15.7 | +/− | +/− | |
| (755 nm) | | | | | | | |
| | | | 1 | 31.4 | + | +/− | |
| | | | 2 | 62.8 | + | ++ | |
| | | | 3 | 94.2 | + | ++ | |
| | | | 4 | 125.6 | + | Not Done | |
| | | | 5 | 157 | + | Not Done | |

The invention contemplates using pulse widths in the 1 microsecond to 50 nanosecond range with fluences between 0.5 and 4 J/cm². In one embodiment the invention contemplates a laser with pulse widths in the 10-50 nanosecond range and fluences between about 0.5 and 1 J/cm². In another embodiment the invention contemplates a laser with a pulse width of about 1 microsecond and a fluence range of about 1-4 J/cm².

The invention further contemplate a system within the aforementioned power ranges that has a high numerical aperture and whose focal point can be set within the sclera pigmentation, about 0.3 mm below the surface, so that the diverging beam will safely enter the eye and cause no damage to the retina, but at the focal plane will be highly effective to destroy the OM. The combination of small exposure spot size and high NA makes the system inherently safe and poses little or no risk of eye injury even when accidentally delivered directly into the pupil.

The invention further contemplates a system that can treat aberrant scleral pigmentation or OM by applying between about 7 mJ and 35 mJ of energy of visible light most preferably with a wavelength in the blue spectrum.

The invention further contemplates an attenuation system for commercially available lasers that will allow for treatment of ocular conditions such as ocular melanosis. In particular, the invention contemplates an attenuation system that provides fluences of 0.5-5 J/cm². In particular, the invention contemplates a contact handpiece, or delivery through a slit lamp that would provide for the fluences stated.

1. A system for treating ocular melanosis comprising a low power laser.

2. The system of claim 1 wherein the laser has a high numerical aperture and whose focal point is set about 0.3 mm below the surface.

3. The system of claim 1 wherein the laser is Q-switched and has a pulse width from about 1 nanosecond to 100 microseconds.

4. The system of claim 3 wherein the pulse width is from about 0.1 microseconds to about 10 microseconds.

5. The system of claim 4 wherein the pulse width is about 1 microsecond.

6. The system of claim 1 wherein the laser emits light in the visible spectrum and has a fluence of between about 0.5 J/cm² and 5 J/cm².

7. The system of claim 2 wherein the laser light is from about 400 nm to about 550 nm.

8. A system for relieving aberrant pigmentation of the sclera of the eye comprising a low power laser.

9. The system of claim 8 wherein the laser has a high numerical aperture and whose focal point is set about 0.3 mm below the surface.

10. The system of claim 8 wherein the laser is Q-switched and has a pulse width from about 1 nanosecond to 100 microseconds.

11. The system of claim 10 wherein the pulse width is from about 0.1 microseconds to about 10 microseconds.

12. The system of claim 11 wherein the pulse width is about 1 microsecond.

13. The system of claim 8 wherein the laser emits light in the visible spectrum and has a fluence of between about 0.5 J/cm² and 5 J/cm².

14. The system of claim 13 wherein the laser light is from about 400 nm to about 550 nm.

15. A method for treating ocular melanosis in an eye comprising the steps of (a) exposing melanocytes in the eye to the light from a low power laser; (b) monitoring the eye for immediate whitening; and (c) terminating exposure of the eye to the laser light prior to the onset of retinal damage.

16. The method of claim 15 wherein the laser has a high numerical aperture and whose focal point is set about 0.3 mm below the surface.

17. The method of claim 15 wherein the laser is Q-switched and has a pulse width from about 1 nanosecond to 100 microseconds.

18. The method of claim 18 wherein the pulse width is from about 0.1 microseconds to about 10 microseconds.

19. The method of claim 18 wherein the pulse width is about 1 microsecond.

20. The method of claim 15 wherein the laser emits light in the visible spectrum and has a fluence of between about 0.5 J/cm² and 5 J/cm².

21. The system of claim 20 wherein the laser emits light having a wavelength of from about 400 nm to about 550 nm.

22. A method for removing aberrant pigmentation in the sclera of an eye comprising the steps of (a) exposing the aberrant pigmentation in the eye to the light from a low power laser; (b) monitoring the eye for immediate whitening; and (c) terminating exposure of the eye to the laser light prior to the onset of retinal damage.

23. The method of claim 22 wherein the laser has a high numerical aperture and whose focal point is set about 0.3 mm below the surface.

24. The method of claim 22 wherein the laser is Q-switched and has a pulse width from about 1 nanosecond to 100 microseconds.

25. The method of claim 24 wherein the pulse width is from about 0.1 microseconds to about 10 microseconds.

26. The method of claim 25 wherein the pulse width is about 1 microsecond.

27. The method of claim 22 wherein the laser emits light in the visible spectrum and has a fluence of between about 0.5 J/cm² and 5 J/cm².

28. The method of claim 27 wherein the laser light is from about 400 nm to about 550 nm.

29. A laser for use in the treatment of ocular melanosis comprising a low power laser.

30. The laser of claim 29 wherein the laser has a high numerical aperture and whose focal point is set about 0.3 mm below the surface.

31. The laser of claim 29 wherein the laser is Q-switched and has a pulse width from about 1 nanosecond to 100 microseconds.

32. The laser of claim 30 wherein the pulse width is from about 0.1 microseconds to about 10 microseconds.

33. The laser of claim 31 wherein the pulse width is about 1 microsecond.

34. The laser of claim 29 wherein the laser emits light in the visible spectrum and has a fluence of between about 0.5 J/cm² and 5 J/cm².

35. The laser of claim 34 wherein the laser light is from about 400 nm to about 550 nm.
